(19) United States
(12) Patent Application Publication
(10) Pub. No.: US 2020/0001274 A1
(43) Pub. Date: Jan. 2, 2020

(54) Palladium Catalysts Supported on Carbon for Hydrogenation of Aromatic Hydrocarbons

(71) Applicants: Simon G. Podkolzin, Hoboken, NJ (US); Tao Chen, Jersey City, NJ (US); Yiteng Zheng, Ridgefield, NJ (US); Muye Yang, Tianjin (CN); Sunitha Rao Tadepalli, Morganville, NJ (US); Geatesh Karunakaran Tampy, Colts, NJ (US); John P. Cherkauskas, JR., Burlington, NJ (US)

(72) Inventors: Simon G. Podkolzin, Hoboken, NJ (US); Tao Chen, Jersey City, NJ (US); Yiteng Zheng, Ridgefield, NJ (US); Muye Yang, Tianjin (CN); Sunitha Rao Tadepalli, Morganville, NJ (US); Geatesh Karunakaran Tampy, Colts, NJ (US); John P. Cherkauskas, JR., Burlington, NJ (US)

(21) Appl. No.: 16/457,665

(22) Filed: Jun. 28, 2019

Related U.S. Application Data
(60) Provisional application No. 62/691,565, filed on Jun. 28, 2018.

(51) Int. Cl. B01J 23/44; B01J 35/00; B01J 37/02; B01J 21/18; B01J 23/50; B01J 23/58; C07C 5/10

(52) U.S. Cl. CPC B01J 23/44; B01J 35/0066; B01J 37/0207; B01J 21/18; B01J 37/0236; C07C 2523/58; B01J 23/58; C07C 5/10; C07C 2523/44; C07C 2521/18; C07C 2523/66; B01J 23/50

(57) ABSTRACT

Provided is a process for preparing partially or fully hydrogenated hydrocarbons through hydrogenation of aromatic hydrocarbons in the presence of a hydrogenation catalyst. The hydrogenation catalyst comprises palladium deposited on carbon with optional acid wash and calcination treatments and with optional additions of silver and/or alkali metals.

### CROSS-REFERENCE TO RELATED APPLICATION

This application claims priority to U.S. Provisional Patent Application Ser. No. 62/691,565 filed Jun. 28, 2018, the entire disclosure of which is incorporated herein by reference.

### FIELD OF THE INVENTION

This invention relates generally to catalysts and methods for the hydrogenation of aromatic hydrocarbons. Aromatic hydrocarbons (hydrocarbons that contain at least one benzene ring) can be fully hydrogenated into alkanes, including cycloalkanes, or partially hydrogenated to intermediate products with one or more carbon-carbon double bonds. More particularly, the invention describes a catalyst with palladium supported on carbon with optional metal additives and methods for the preparation of this catalyst.

### BACKGROUND OF THE INVENTION

In multiple industrial chemical applications, such as the production of diesel fuels, jet fuels and the production of chemicals, it is desirable to partially or fully hydrogenate aromatic hydrocarbons. The invention describes a process of preparing improved catalysts for hydrogenation of aromatic hydrocarbons. Methods of using these catalysts are also described.

In the production of diesel fuels, hydrogenation of aromatic hydrocarbons is desirable because it increases the hydrogen to carbon ratio and, therefore, allows the fuel to burn cleaner (lower carbon dioxide emissions) and improves the cetane number of the fuel (improves its quality). In the production of jet fuels, hydrogenation of aromatic hydrocarbons is desirable because it increases the hydrogen to carbon ratio and, therefore, makes the fuel more thermally stable and improves the smoke point of the fuel (allows the fuel to burn cleaner with lower carbo dioxide emissions).

In the production of specialty chemicals, for example in the production of flavors and fragrances, it is desirable to partially and selectively hydrogenate aromatic hydrocarbons into less unsaturated hydrocarbons for functionalization of the remaining carbon-carbon double bonds in the obtained less unsaturated hydrocarbons. For example, it is desirable to partially hydrogenate 1,1,2,3,3-pentamethyl indane (PMI) to 1,1,2,3,3-pentamethyl-tetrahydro indane (THPMI) so that the remaining carbon-carbon double bond in THPMI can be functionalized. In this case, it is desirable to avoid complete hydrogenation of the benzene ring in PMI with the formation of 1,1,2,3,3-pentamethyl-hexahydro indane (HHPMI). In addition, tetralin and its hydrogenated product decalin, are used as solvents in dry cleaning of clothes and in the production of paints, fats, resins, lacquers, varnishes, shoe creams, floor waxes and other consumer products. In the production of commodity chemicals, benzene and its substituted derivatives, for example toluene, xylenes, etc., are hydrogenated to cyclohexane and its substituted derivatives in the production of ketones and aldehydes that are used in the production of other chemicals, including monomers for the production of Nylon 6 and Nylon 6,6. Aniline, a nitrogen-containing hydrocarbon with a benzene ring, is hydrogenated to cyclohexylamine in the production of emulsifiers, antioxidants and artificial sweeteners. Aniline is also hydrogenated to dicyclohexylamine in the production of vulcanization accelerators, pesticides and corrosion inhibitors.

In environmental applications, such purification of wastewater, it is important to hydrogenate and preferably decompose aromatic hydrocarbons. For example, it is important to treat and degrade 2, 4-dinitroanisole (DNAN), a nitrogen-containing hydrocarbon with a benzene ring, which is present in the wastewater generated in the production of explosives.

It is generally desirable to improve the catalytic activity, allowing to reduce the amount of the catalyst, reduce the time required for the hydrogenation reaction to reach a desired conversion target, reduce the temperature required for the reaction and/or reduce the size of the reactor. Improved catalytic activity is, therefore, advantageous for the industrial efficiency of performing hydrogenation reactions. In the production of partially hydrogenated hydrocarbons, higher catalytic selectivity allows to obtain higher yields of the desirable products, while lowering the production of undesirable byproducts and, therefore, higher catalytic selectivity is advantageous for the industrial efficiency.

In commercial operations, nickel and platinum supported on alumina or Raney nickel without a support are used as catalysts for hydrogenation of aromatic hydrocarbons. In addition, palladium supported on an activated carbon or alumina are also used. The concentration of palladium varies between 1 and 5 wt % with a typical concentration of 5 wt % for use as a powder in a slurry reactor and between 0.1 and 1 wt % with a typical concentration of 0.5 wt % for use as extrudates, spheres, tablets or granules in a fixed bed reactor.

### SUMMARY OF THE INVENTION

The invention relates to a palladium catalyst exhibiting improved activity in hydrogenation of aromatic hydrocarbons. In particular, palladium is deposited on a carbon support. In one embodiment, the catalyst comprises from about 0.1 to about 5 wt % of palladium deposited on carbon.

In one embodiment, the invention relates to optional treatments that further improve the activity of catalysts with palladium supported on carbon in hydrogenation of aromatic hydrocarbons. In other embodiments, one or more of the following catalyst treatments is performed: (a) to wash the carbon with an acid prior to the use of this carbon as the support for palladium, (b) to calcine (treat with oxygen at an elevated temperature) the carbon support prior to the metal deposition, (c) to avoid catalyst calcination after the metal deposition, and (d) to avoid catalyst reduction pretreatment (treatment with hydrogen at an elevated temperature prior to a hydrocarbon hydrogenation reaction).

In one embodiment, silver and/or alkali metals (for example, sodium or potassium) are added to the composition of a catalyst with palladium deposited on carbon so as to improve selectivity to partially hydrogenated products. The molar ratio of palladium to an additive is in the range from about 1 to about 12.

In one embodiment, the catalysts comprising palladium deposited on carbon with optional silver and/or alkali metals can be used for hydrogenation of hydrocarbons other than aromatic hydrocarbons. Moreover, since a catalyst increases the rates of forward and reverse reactions, the catalysts can be used for the reverse reactions: dehydrogenation of hydrocarbons to the corresponding unsaturated hydrocarbons.

### DETAILED DESCRIPTION OF EXEMPLARY EMBODIMENTS OF THE INVENTION

In one embodiment, the invention provides a method for producing partially or fully hydrogenated hydrocarbons by reacting aromatic hydrocarbons with a hydrogen-containing gas in the presence of a catalyst that comprises palladium supported on carbon.

In accordance with one embodiment, a palladium catalyst exhibits improved activity in hydrogenation of aromatic hydrocarbons when palladium is deposited on a carbon support, compared to other possible supports, for example, silica, alumina, silica-alumina and titania. The catalyst comprises from about 0.1 to about 5 wt % of palladium deposited on a carbon. An additional advantage of a carbon support is that palladium and other deposited metals can be easily recovered by simply burning off the carbon, whereas more complex methods for metal recovery are required for other support types, such as silica, alumina, silica-alumina and titania.

In one embodiment, a palladium catalyst with palladium deposited on a carbon support is prepared dissolving a precursor, such as palladium(II) nitrate hydrate, Pd(NO₃)₂.xH₂O (Sigma Aldrich 205761-2G), in deionized water to make a single solution. The solution was then deposited onto a carbon support, such as acid-washed activated carbon (e.g., Cabot Norit SX 2), using the incipient wetness impregnation method. The solution was added dropwise to the support with continuous mixing and stirring. After the metal deposition, the sample was dried in an oven in static air at a suitable temperature (e.g., 120° C.) for a suitable time period (e.g., overnight or approximately 12 hours).

### Example 1: Use of Carbon as a Support for Pd Catalysts Compared to Other Supports, Such as Silica, Alumina, Silica-Alumina and Titania

### Catalyst 1

5 wt % Pd/silica was synthesized by dissolving the precursor, palladium(II) nitrate hydrate, Pd(NO₃)₂.xH₂O (Sigma Aldrich 205761-2G), in deionized water to make a single solution. The solution was then deposited onto a support using the incipient wetness impregnation method. For Catalyst 1, the support was silica (Saint-Gobain NorPro SS 61138). The solution was added dropwise to the support with continuous mixing and stirring. After the metal deposition, the sample was dried in an oven in static air at 120° C. overnight (˜12 hours) and used for testing without any additional pretreatment (without calcination or reduction).

### Catalyst 2

5 wt % Pd/titania was synthesized using the same procedure as Catalyst 1, with the exception that the support was titania (Saint-Gobain NorPro ST 61120).

### Catalyst 3

5 wt % Pd/fumed silica was synthesized using the same procedure as Catalyst 1, with the exception that the support was fumed silica (Cabot CAB-O-SIL HS-5).

### Catalyst 4

5 wt % Pd/alumina was synthesized using the same procedure as Catalyst 1, with the exception that the support was alumina (Saint-Gobain NorPro SA 6175).

### Catalyst 5

5 wt % Pd/silica-alumina was synthesized using the same procedure as Catalyst 1, with the exception that the support was silica-alumina (Saint-Gobain NorPro SS

61155).

Catalyst 6

5 wt % Pd/carbon was synthesized using the same procedure as Catalyst 1, with the exception that the support was acid-washed activated carbon (Cabot Norit SX 2).

Catalysts 1-6 were tested by hydrogenating 1,1,2,3,3-pentamethyl indane (PMI) to 1,1,2,3,3-pentamethyl-tetrahydro indane (THPMI) and further to 1,1,2,3,3-pentamethyl-hexahydro indane (HHPMI) using the following protocol:

1. A 300 mL Parr reactor was loaded with 40.0 g of PMI and 120.0 g of decahydronaphthalene (decalin) (with a PMI to decalin mass ratio of 1 to 3). 1 wt % of a solid catalyst (0.40 g) was added to the liquid.

2. The reactor was flushed with N₂ twice and checked for leaks.

3. The reactor was filled with H₂ at 100 psi and checked for leaks.

4. The reactor H₂ pressure was increased to 400 psig, and mixing started with an agitation speed of 700 rpm.

5. Temperature was raised to the desired testing temperature of 200° C. and held constant for the duration of the test.

6. After reaching the desired testing temperature, the reactor H₂ pressure was increased to 650 psig. This point of the pressure increase to 650 psig was taken as zero time on stream.

7. Liquid samples from the reactor were collected every 30 min and analyzed using a gas chromatograph (GC) equipped with a flame ionization detector and a Carbowax HP-5 column.

8. The temperature profile for the GC oven was as follows:
a) Temperature was held constant at 50° C. for 1 min.
b) The temperature was ramped to 80° C. at a rate of 15° C./min and held for 5 min.
c) The temperature was increased to 180° C. with a ramp rate of 20° C./min and held constant until the end of the run.

9. The pressure of the reactor was maintained at 650 psig using an external gas burette equipped with a high-pressure regulator.

TABLE 1
PMI conversion (wt %) to THPMI and HHPMI at 200° C. and 650
psig hydrogen pressure in a batch reactor as a function of reaction
time for 5 wt % Pd supported on different materials.

| | Catalyst | Catalyst | Catalyst 3 | | Catalyst 5 | |
|---|---|---|---|---|---|---|
| Time, | 1 | 2 | fumed | Catalyst 4 | silica- | Catalyst 6 |
| h | silica | titania | silica | alumina | alumina | carbon |
| 0.5 | | | | 9 | 8 | 4 |
| 1.0 | 7 | 16 | | 15 | 15 | 12 |
| 1.5 | 7 | 16 | 27 | 19 | 17 | 28 |
| 2.0 | 8 | 20 | 31 | 24 | 23 | 44 |
| 2.5 | 9 | 23 | 36 | 26 | 28 | 63 |
| 3.0 | 10 | 28 | 38 | 30 | 34 | 79 |
| 3.5 | 12 | 32 | 42 | 34 | 40 | |
| 4.0 | 14 | 36 | 49 | 38 | 43 | |
| 4.5 | 15 | 39 | 51 | 40 | 49 | |
| 5.0 | 17 | 41 | 54 | 45 | 54 | |
| 5.5 | 18 | | 59 | 47 | 59 | |
| 6.0 | | | 60 | 51 | | |
| 6.5 | | | | 56 | | |
| 7.0 | | | | 58 | | |

The results in Table 1 demonstrate that palladium is more catalytically active (has higher PMI conversion as a function of time after 1 hour) when carbon is used as a support (Catalyst 6).

In another embodiment, the invention relates to optional treatments that further improve the activity of catalysts with palladium supported on carbon in hydrogenation of aromatic hydrocarbons. It is advantageous to optionally perform one or more of the following catalyst treatments: (a) to wash the carbon with an acid prior to the use of this carbon as the support for palladium, (b) to calcine (treat with oxygen at an elevated temperature) the carbon support prior to the metal deposition, (c) to avoid catalyst calcination after the metal deposition, and (d) to avoid catalyst reduction (treatment with hydrogen at an elevated temperature).

### Example 2A: Use an Acid-Washed Carbon Support

### Catalyst 6

5 wt % Pd/carbon (acid-washed) was the same Catalyst 6 described in Example 1.

### Catalyst 7

5 wt % Pd/carbon (non-acid-washed) was synthesized using the same procedure as Catalyst 6, with the exception that the support used was non-acid-washed activated carbon (Cabot Norit SX 1G).

Catalysts 6 and 7 were tested using the same protocol described in Example 1.

TABLE 2A
PMI conversion (wt %) to THMPI and HHPMI and selectivity
(wt %) to THPMI at 200° C. and 650 psig hydrogen
pressure in a batch reactor as a function of reaction time for
5 wt % Pd supported on acid-washed and non-acid-washed carbon.

| | Catalyst 6 | | Catalyst 7 | |
|---|---|---|---|---|
| | acid-washed carbon | | non-acid-washed carbon | |
| Time, h | Conversion | Selectivity | Conversion | Selectivity |
| 0.5 | 4 | 76 | 25 | 55 |
| 1.0 | 12 | 81 | 28 | 53 |
| 1.5 | 28 | 77 | 33 | 48 |
| 2.0 | 44 | 71 | 39 | 43 |
| 2.5 | 63 | 61 | 50 | 35 |
| 3.0 | 79 | 49 | | |

The results in Table 2A demonstrate that palladium exhibits higher activity and selectivity when it is supported on the acid-washed carbon (Catalyst 6) compared to the non-acid-washed carbon (Catalyst 7).

### Example 2B: Calcination of the Carbon Support Prior to the Metal Deposition

### Catalyst 6

5 wt % Pd/carbon (without support calcination) was the same Catalyst 6 described in Example 1.

### Catalyst 8

5 wt % Pd/carbon (with support calcination) was synthesized by dissolving the precursor, palladium(II) nitrate hydrate, Pd(NO₃)₂.xH₂O (Sigma Aldrich 205761-2G), in deionized water to make a single solution. The solution was then deposited onto a support using the incipient wetness impregnation method. For Catalyst 8, the support was acid-washed activated carbon (Cabot Norit Plus). This carbon support was subjected to a calcination treatment prior to the palladium deposition. The carbon support calcination treatment was performed in a furnace in the presence of static air (the air was not flowing) by raising the temperature at 10° C./min to 350° C., holding at this temperature for 2 hours and then cooling down to room temperature. The palladium solution was added dropwise to the support with continuous mixing and stirring. After the metal deposition, the sample was dried in an oven in static air at 120° C. overnight (˜12 hours) and used for testing without any additional pretreatment (without reduction).

Catalysts 6 and 8 were tested using the same protocol described in Example 1, with the exception that the testing temperature was 180° C.

TABLE 2B
PMI conversion (wt %) to THPMI and HHPMI and selectivity
(wt %) to THPMI at 180° C. and 650 psig hydrogen
pressure in a batch reactor as a function of reaction time for
wt % Pd supported on carbon with and without calcination.

| | Catalyst 6 | | Catalyst 8 | |
|---|---|---|---|---|
| | uncalcined carbon | | calcined carbon | |
| Time, h | Conversion | Selectivity | Conversion | Selectivity |
| 0.5 | 12 | 50 | 9 | 85 |
| 1.0 | 15 | 58 | 10 | 84 |
| 1.5 | 19 | 62 | 15 | 85 |
| 2.0 | 24 | 61 | 21 | 84 |
| 2.5 | 28 | 56 | 27 | 81 |
| 3.0 | 35 | 49 | 41 | 73 |
| 3.5 | | | 45 | 71 |
| 4.0 | | | 55 | 62 |
| 4.5 | | | 61 | 59 |
| 5.0 | | | 68 | 50 |
| 5.5 | | | 76 | 43 |

The results in Table 2B demonstrate that palladium exhibits higher activity after 2.5 hours and improved selectivity for the duration of the run when it is supported on the calcined carbon (Catalyst 8) compared to the uncalcined carbon (Catalyst 6).

### Example 2C: Avoiding Catalyst Calcination after the Metal Deposition

### Catalyst 6

5 wt % Pd/carbon (acid-washed, uncalcined) was the same Catalyst 6 described in Example 1.

### Catalyst 9

5 wt % Pd/carbon (acid-washed, calcined) was synthesized by dissolving the precursor, palladium(II) nitrate hydrate, Pd(NO₃)₂.xH₂O (Sigma Aldrich 205761-2G), in deionized water to make a single solution. The solution was then deposited onto a support using the incipient wetness impregnation method. For Catalyst 9, the support was acid-washed activated carbon (Cabot Norit Plus) that was subjected to a calcination treatment after the palladium deposition. The palladium solution was added dropwise to the support with continuous mixing and stirring. After the palladium deposition, the sample was dried in an oven in static air at 120° C. overnight (˜12 hours). The catalyst calcination was performed in a Micromeritics furnace in the presence of air flow at 50 sccm by raising the temperature at 2° C./min to 120° C. and then ramping at 10° C./min to 350° C., holding at this temperature for 2 hours and then cooling down to room temperature.

Catalysts 6 and 9 were tested using the same protocol described in Example 1.

TABLE 2C
PMI conversion (wt %) to THMPI and HHPMI and selectivity
(wt %) to THPMI at 200° C. and 650 psig hydrogen
pressure in a batch reactor as a function of reaction
time for calcined and uncalcined 5 wt % Pd/C catalysts.

| | Catalyst 6 | | Catalyst 9 | |
|---|---|---|---|---|
| | uncalcined Pd/C | | calcined Pd/C | |
| Time, h | Conversion | Selectivity | Conversion | Selectivity |
| 0.5 | 4 | 76 | 20 | 64 |
| 1.0 | 12 | 81 | 23 | 74 |
| 1.5 | 28 | 77 | 29 | 78 |
| 2.0 | 44 | 71 | 37 | 76 |
| 2.5 | 63 | 61 | 46 | 72 |
| 3.0 | 79 | 49 | 51 | 70 |
| 3.5 | | | 56 | 68 |
| 4.0 | | | 62 | 64 |

The results in Table 2C demonstrate that calcination of Pd/C catalysts generally reduces the catalyst activity. The catalytic activity of the uncalcined catalyst (Catalyst 6) is higher after 1.5 hours on stream than that of the analogous calcined catalyst (Catalyst 9). It is, therefore, advantageous to avoid catalyst calcination after the metal deposition.

### Example 2D: Avoiding Catalyst Reduction

### Catalyst 10

5 wt % Pd/silica-alumina (calcined, reduced) was synthesized by dissolving the precursor, palladium(II) nitrate hydrate, Pd(NO₃)₂.xH₂O (Sigma Aldrich 205761-2G), in deionized water to make a single solution. The solution was then deposited onto a support using the incipient wetness impregnation method. For Catalyst 10, the support was silica-alumina (Saint-Gobain NorPro SS 61155 SiO₂—Al₂O₃). The solution was added dropwise to the support with continuous mixing and stirring. After the metal deposition, the sample was dried in an oven in static air at 120° C. overnight (˜12 hours). The catalyst was subjected to a calcination treatment, which was performed in a Micromeritics furnace in the presence of air flow at 50 sccm by raising the temperature at 2° C./min to 120° C. then ramping at 10° C./min to 350° C., holding at this temperature for 2 hours and then cooling down to room temperature. The catalyst was subjected to a reduction treatment after the calcination treatment. The catalyst was reduced in a 50 sccm flow of 10 mol % H₂/He at 150° C. for 2 hours and then cooled to room temperature.

### Catalyst 11

5 wt % Pd/silica-alumina (calcined, unreduced) was synthesized using the same procedure as Catalyst 10, with the exception that the catalyst was not subjected to a reduction treatment after the calcination treatment.

Catalysts 10 and 11 were tested using the same protocol as in Example 1.

TABLE 2D
PMI conversion (wt %) to THMPI and HHPMI and selectivity
(wt %) to THPMI at 200° C. and 650 psig hydrogen
pressure in a batch reactor as a function of reaction time for
reduced and unreduced 5 wt % Pd/SiO₂—Al₂O₃ catalysts.

| | Catalyst 10 | | Catalyst 11 | |
|---|---|---|---|---|
| | reduced | | unreduced | |
| | Pd/silica-alumina | | Pd/silica-alumina | |
| Time, h | Conversion | Selectivity | Conversion | Selectivity |
| 1.5 | 10 | 74 | 19 | 75 |
| 2.0 | 13 | 76 | 22 | 76 |
| 2.5 | 15 | 75 | 26 | 73 |
| 3.0 | 19 | 75 | 31 | 71 |
| 3.5 | 25 | 71 | 37 | 69 |
| 4.0 | 28 | 69 | 40 | 68 |
| 4.5 | 32 | 66 | 45 | 66 |
| 5.0 | 36 | 65 | 50 | 64 |
| 5.5 | 40 | 61 | 55 | 61 |
| 6.0 | 42 | 60 | | |
| 6.5 | 47 | 59 | | |
| 7.0 | 50 | 56 | | |

The results in Table 2D demonstrate that the reduction of the catalyst prior to the start of the hydrocarbon hydrogenation reaction decreases the catalyst activity. It is, therefore, advantageous to avoid catalyst reduction pretreatment.

In another embodiment, silver and/or alkali metals (for example, sodium or potassium) are added to the composition of a catalyst with palladium deposited on carbon advantageously improves selectivity to partially hydrogenated products. The molar ratio of palladium to an additive is in the range from about 1 to about 12.

### Example 3: Adding Silver (Aq) and/or Alkali Metals (for Example, Na or K) to Pd

### Catalyst 8

5 wt % Pd/carbon was the same Catalyst 8 described in Example 2B.

### Catalyst 12

5 wt % Pd—K (molar ratio 6:1 of Pd to K)/carbon was synthesized using a calcined activated carbon (Cabot Norit SX Plus) as the support. The carbon calcination was performed in a furnace in the presence of static air (the air was not flowing) by raising the temperature at 10° C./min to 350° C., holding at this temperature for 2 hours and then cooling down to room temperature. The first precursor, palladium(II) nitrate hydrate, and the second precursor, potassium nitrate (Sigma Aldrich P8384-500G), were dissolved in deionized water to make a single solution. The solution was then deposited onto the support using the incipient wetness impregnation method. The solution was added dropwise to the support with continuous mixing and stirring. After the metal deposition, the sample was dried in an oven in static air at 120° C. overnight (˜12 hours) and used for testing without any additional pretreatment (without catalyst calcination or reduction).

### Catalyst 13

5 wt % Pd—Na (molar ratio 3:1 of Pd to Na)/carbon was synthesized using the same procedure as Catalyst 12, with the exception that the second precursor was sodium nitrate (Sigma Aldrich 55506-500G).

### Catalyst 14

5 wt % Pd—Ag (molar ratio 6:1 of Pd to Ag)/carbon was synthesized using the same procedure as Catalyst 12, with the exception that the second precursor was silver nitrate (Sigma Aldrich 209139-25G).

Catalysts 8, 12, 13, and 14 were tested using the same protocol described in Example 1, with the exception that the testing temperature was 180° C.

TABLE 3
PMI conversion (wt %) to THPMI and HHPMI and selectivity (wt %) to THPMI
at 180° C. and 650 psig hydrogen pressure in a batch reactor as a function of reaction
time for 5 wt % Pd/C and 5 wt % Pd—Me/C, where Me is a second metal: K, Na or Ag.

| | Catalyst 8 | | Catalyst 12 | | Catalyst 13 | | Catalyst 14 | |
|---|---|---|---|---|---|---|---|---|
| | Pd/C | | Pd—K (6:1)/C | | Pd—Na (3:1)/C | | Pd—Ag (6:1)/C | |
| Time, h | Conversion | Selectivity | Conversion | Selectivity | Conversion | Selectivity | Conversion | Selectivity |
| 1.0 | 10 | 84 | 9 | 94 | 1 | 89 | 9 | 83 |
| 1.5 | 15 | 85 | 25 | 85 | 2 | 91 | 12 | 88 |
| 2.0 | 21 | 84 | 31 | 83 | 4 | 90 | 17 | 89 |
| 2.5 | 27 | 81 | 36 | 82 | 4 | 90 | 20 | 88 |
| 3.0 | 41 | 73 | 40 | 79 | 6 | 90 | 25 | 88 |
| 3.5 | 45 | 71 | 48 | 76 | 8 | 89 | 32 | 86 |
| 4.0 | 55 | 62 | 56 | 71 | | | 37 | 85 |
| 4.5 | 61 | 59 | 67 | 64 | | | 43 | 84 |
| 5.0 | 68 | 50 | 72 | 60 | | | 46 | 83 |
| 5.5 | 76 | 43 | 77 | 46 | | | 46 | 81 |

The results in Table 3 demonstrate that the addition of a second metal (potassium, sodium or silver) increases the catalyst selectivity to the partially hydrogenated product.

In one embodiment, catalysts comprising palladium deposited on carbon with optional silver and/or alkali metals may be used for hydrogenation of hydrocarbons other than aromatic hydrocarbons. In other embodiments, since a catalyst increases the rates of forward and reverse reactions, the catalysts can be used for the reverse reactions: dehydrogenation of hydrocarbons to the corresponding unsaturated hydrocarbons.

It should be understood that the embodiments described herein are merely exemplary in nature and that a person skilled in the art may make many variations and modifications thereto without departing from the scope of the present invention. All such variations and modifications, including those discussed above, are intended to be included within the scope of the invention.

We claim:

1. A chemical catalyst, comprising an acid-washed carbon base and palladium deposited on said carbon base.

2. The chemical catalyst of claim 1, wherein said carbon base is an activated carbon base.

3. The chemical catalyst of claim 1, wherein said carbon base is calcinated before said palladium is deposited thereon.

4. The chemical catalyst of claim 1, wherein said catalyst comprises from about 0.1 to about 5 weight percentage of palladium.

5. The chemical catalyst of claim 1, further comprising a metal additive deposited on said carbon base with said palladium.

6. The chemical catalyst of claim 5, wherein the molar ratio of said palladium to said metal additive is in a range of from 1:1 to 12:1.

7. The chemical catalyst of claim 5, wherein said metal additive comprises a metal selected from the group consisting of alkali metals and silver.

8. A method of making a chemical catalyst, comprising the steps of:
(i) dissolving a first precursor in deionized water to form a solution;
(ii) depositing said solution onto an acid-washed carbon base; and
(iii) drying said carbon base in the presence of static air.

9. The method of claim 8, wherein step (ii) is conducted according to the incipient wetness method.

10. The method of claim 8, wherein said carbon base is an activated carbon base.

11. The method of claim 8, further comprising the step of calcining said carbon base prior to the performance of step (ii).

12. The method of claim 11, wherein no calcination treatment is applied to said carbon base following the performance of step (ii).

13. The method of claim 11, wherein said calcining step involves subjecting said carbon base to a heat-treatment process in the presence of static air.

14. The method of claim 8, wherein said carbon base is not subjected to reduction treatment following the performance of step (ii).

15. The method of claim 8, wherein no calcination treatment is applied to said carbon base following the performance of step (ii).

16. The method of claim 8, wherein said first precursor comprises palladium.

17. The method of claim 8, wherein said first precursor is palladium(II) nitrate hydrate.

18. The method of claim 8, further comprising the step of adding a second precursor to said solution before the performance of step (ii).

19. The method of claim 18, wherein said second precursor comprises a metal selected from the group consisting of alkali metals and silver.

20. The method of claim 19, wherein said first precursor comprises palladium and wherein the molar ratio of said palladium to said metal is in a range of from 1:1 to 12:1 following the performance of step (iii).

21. The method of claim 18, wherein said second precursor is selected from the group consisting of silver nitrate, sodium nitrate and potassium nitrate.

22. A chemical catalyst, comprising a carbon base; palladium deposited on said carbon base; and a metal additive deposited on said carbon base in combination with said palladium.

23. The chemical catalyst of claim 22, wherein said carbon base is an activated carbon base.

24. The chemical catalyst of claim 22, wherein said carbon base is calcinated before said palladium is deposited thereon.

25. The chemical catalyst of claim 24, wherein said carbon base has been acid-washed before said palladium is deposited on said carbon base.

26. The chemical catalyst of claim 22, wherein the molar ratio of said palladium to said metal additive is in a range of from 1:1 to 12:1.

27. The chemical catalyst of claim 22, wherein said metal additive comprises a metal selected from the group consisting of alkali metals and silver.

28. A process for preparing partially or fully hydrogenated hydrocarbons, said process comprising the step of hydrogenating an aromatic hydrocarbon in the presence of a hydrogenation catalyst, wherein said catalyst comprises an acid-washed carbon base and palladium.

29. The process of claim 28, wherein said acid-washed carbon base is an activated carbon base.

30. The process of claim 28, wherein said catalyst comprises from about 0.1 to about 5 weight percentage of palladium.

31. The process of claim 28, further comprising the step of depositing said palladium on said acid-washed carbon base.

32. The process of claim 31, further comprising the step calcinating said acid-washed carbon base before depositing said palladium thereon.

33. The process of claim 28, further comprising the step of depositing a metal additive on said acid-washed carbon base with said palladium.

34. The process of claim 33, wherein the molar ratio of said palladium to said metal additive is in a range of from 1:1 to 12:1.

35. The process of claim 33, wherein said metal additive comprises a metal selected from the group consisting of alkali metals and silver.
